(19) United States
(12) Patent Application Publication
(10) Pub. No.: US 2019/0358160 A1
(43) Pub. Date: Nov. 28, 2019

(54) SUB-MICRON EMULSIONS

(71) Applicant: Formulytica Pty Ltd, Mulgrave (AU)

(72) Inventors: Richard Buchta, Mulgrave (AU); Robert Houlden, Mulgrave (AU); Biljana Roughan, Mulgrave (AU); Sasaros Anuvechsirikiat, Mulgrave (AU)

(73) Assignee: Formulytica Pty Ltd, Mulgrave (AU)

(21) Appl. No.: 16/407,740

(22) Filed: May 9, 2019

Related U.S. Application Data
(60) Provisional application No. 62/670,480, filed on May 11, 2018.

(51) Int. Cl. A61K 9/107; A61K 47/10; A61K 47/44; A61K 47/18; A61K 47/12

(52) U.S. Cl. CPC A61K 9/1075; A61K 47/10; A61K 45/06; A61K 47/183; A61K 47/12; A61K 47/44

(57) ABSTRACT

Sub-micron emulsions and processes for their preparation are disclosed. In particular, sub-micron emulsion compositions containing pharmaceutically active ingredients, including delivery vehicles, such as foams, are disclosed. The emulsion compositions and foams may advantageously deliver pharmaceutically active ingredients, such as, for example, corticosteroids. The emulsions maintain stability over extended periods of time.

### FIELD

The present disclosure relates to sub-micron emulsions and processes for their preparation. In particular, the disclosure relates to sub-micron emulsion compositions containing pharmaceutically active ingredients, and to delivery vehicles, such as foams, comprising the sub-micron emulsion compositions. The emulsion compositions and foams may advantageously deliver pharmaceutically active ingredients.

### BACKGROUND

Emulsion compositions comprising sub-micron particles have previously been applied to the delivery of active pharmaceutical ingredients across the skin barrier. As particles become smaller their ability to cross the skin barrier is enhanced and, further, as their surface to volume ratio increases potential efficacy increases.

Sub-micron emulsions have also been utilized in the topical delivery of active pharmaceutical ingredients where the active pharmaceutical ingredient is insoluble or substantially insoluble in water.

However, prior art processes for the preparation of sub-micron emulsions are often complex, requiring multiple surfactants differing in their hydrophilic-lipophilic balance (HLB) and often requiring separate addition of the active pharmaceutical ingredient in a solvent once the emulsion is formed.

For example, United States application publication No. 2006/0057168 describes a process for the preparation of oil in water microemulsions or sub-micron emulsion compositions, in particular oil in water microemulsion or sub-micron emulsion foam compositions. These sub-micron or microemulsion foams have a comparatively high oil content and require both hydrophilic and lipophilic surfactants. The publication also teaches that propylene glycol, which is used as solvent for an active pharmaceutical ingredient, is disruptive of emulsion formulations and further teaches the requirement that propylene glycol be added after formation of the emulsion.

The stability of an emulsion over time is also critical to its useful application in cosmetic or therapeutic applications. Further, long term stability of the active pharmaceutical ingredient is also of high importance.

In view of the foregoing it would be desirable to provide sub-micron emulsions that are stable for long periods of time and to provide processes for their preparation that are less complex to prior art processes.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

### SUMMARY

The present disclosure is directed to an oil in water sub-micron emulsion having a water phase and an oil phase, said emulsion comprising water, at least one oil, optionally at least one hydroxyl containing organic solvent, and at least one hydrophilic surfactant having an HLB greater than about 10. The present disclosure advantageously provides for a low oil and a low surfactant content in an oil in water sub-micron emulsion. The present disclosure also provides an oil in water sub-micron emulsion composition having a water phase and an oil phase, said composition comprising at least one active pharmaceutical ingredient, water, at least one oil, optionally at least one hydroxyl containing organic solvent, and at least one hydrophilic surfactant having an HLB greater than about 10. The present disclosure also provides an oil in water sub-micron emulsion aerosol foam comprising water, at least one oil, optionally at least one hydroxyl containing organic solvent, and at least one hydrophilic surfactant having an HLB greater than about 10. The present disclosure further provides an oil in water sub-micron emulsion aerosol foam composition comprising at least one active pharmaceutical ingredient, water, at least one oil, optionally at least one hydroxyl containing organic solvent, and at least one hydrophilic surfactant having an HLB greater than about 10.

The present sub-micron emulsions and sub-micron emulsion compositions are stable over long periods of time and are advantageously straightforward to prepare utilizing a minimum number of components.

In one aspect the present disclosure provides an oil in water sub-micron emulsion, said emulsion comprising an oil phase dispersed throughout a water phase, said oil in water emulsion comprising:
(a) at least one oil;
(b) at least one hydrophilic surfactant having an HLB greater than about 10;
(c) optionally at least one hydroxyl containing organic solvent; and
(d) water;

wherein the at least one surfactant is miscible both with water and the at least one hydroxyl containing organic solvent.

In another aspect the present disclosure provides an oil in water sub-micron emulsion composition, said composition comprising:
(a) at least one active pharmaceutical ingredient; and
(b) an oil in water sub-micron emulsion comprising an oil phase dispersed throughout a water phase, said oil in water sub-micron emulsion comprising:
(i) at least one oil;
(ii) at least one hydrophilic surfactant having an HLB greater than about 10;
(iii) optionally at least one hydroxyl containing organic solvent; and
(iv) water;

wherein the at least one surfactant is miscible both with water and the at least one hydroxyl containing organic solvent.

In another aspect the present disclosure provides an oil in water sub-micron emulsion aerosol foam comprising an oil phase dispersed throughout a water phase, said foam comprising:
(a) at least one oil;
(b) at least one hydrophilic surfactant having an HLB greater than about 10;
(c) optionally at least one hydroxyl containing organic solvent; and
(d) water;

wherein the at least one surfactant is miscible both with water and the at least one hydroxyl containing organic solvent.

In another aspect the present disclosure provides an oil in water sub-micron emulsion aerosol foam composition, said composition comprising:
(a) at least one active pharmaceutical ingredient; and
(b) an oil in water sub-micron emulsion foam comprising an oil phase dispersed throughout a water phase, said oil in water sub-micron emulsion foam composition comprising:
(i) at least one oil;
(ii) at least one hydrophilic surfactant having an HLB greater about 10;
(iii) optionally at least one hydroxyl containing organic solvent; and
(iv) water;

wherein the at least one surfactant is miscible both with water and the at least one hydroxyl containing organic solvent.

In another aspect the present disclosure provides a process for the preparation of an oil in water sub-micron emulsion, comprising water, at least one oil, at least one hydrophilic surfactant having an HLB greater than about 10 and optionally at least one hydroxyl containing organic solvent, the process comprising at least the steps of heating a mixture of at least, water, at least one oil, and at least one hydrophilic surfactant, to a temperature above the cloud point of the hydrophilic surfactant and subsequently cooling the mixture to form an oil in water sub-micron emulsion.

The process may not include a step of phase inversion from an oil in water sub-micron emulsion to a water in oil sub-micron emulsion or a step of phase inversion from a water in oil sub-micron emulsion to an oil in water sub-micron emulsion.

According to another aspect, the present disclosure provides a process for the preparation of an oil in water sub-micron emulsion, comprising water, at least one oil, at least one hydrophilic surfactant having an HLB greater than about 10 and optionally at least one hydroxyl containing organic solvent, the process comprising:
(a) admixing at least one oil, at least one hydrophilic surfactant having an HLB greater than about 10, and water;
(b) heating the mixture of step a) to a temperature above the cloud point of the hydrophilic surfactant so as to form a bicontinuous emulsion;
(c) cooling the bicontinuous emulsion to form an oil in water sub-micron emulsion; and
(d) optionally adding at least one hydroxyl containing organic solvent to the sub-micron emulsion.

The at least one hydroxyl containing solvent may be at or above ambient temperature when added to the cooled emulsion.

According to another aspect, the present disclosure provides a process for the preparation of an oil in water sub-micron emulsion composition, comprising at least one active pharmaceutical ingredient, water, at least one oil, at least one hydrophilic surfactant having an HLB greater than about 10 and optionally at least one hydroxyl containing organic solvent, the process comprising:
(a) admixing at least one oil, at least one hydrophilic surfactant having an HLB greater than about 10 and water;
(b) heating the mixture of step a) to a temperature above the cloud point of the hydrophilic surfactant so as to form a bicontinuous emulsion;
(c) cooling the bicontinuous emulsion to form an oil in water sub-micron emulsion;
(d) adding at least one active pharmaceutical ingredient optionally dissolved in at least one hydroxyl containing organic solvent to the sub-micron emulsion.

The at least one hydroxyl containing solvent may be at or above ambient temperature when added to the cooled emulsion.

According to another aspect, the present disclosure provides a process for the preparation of an oil in water sub-micron emulsion, comprising water, at least one oil, at least one hydrophilic surfactant having an HLB greater than about 10 and optionally at least one hydroxyl containing organic solvent, the process comprising:
(a) heating a mixture of at least one oil, at least one hydrophilic surfactant having an HLB greater than about 10 and optionally at least one hydroxyl containing organic solvent to a temperature above 25° C.;
(b) heating water to a temperature above 25° C.;
(c) combining the heated water with the heated oil mixture;
(d) further heating the mixture of step c) to a temperature above the cloud point of the hydrophilic surfactant so as to form a bicontinuous emulsion;
(e) cooling the bicontinuous emulsion to form an oil in water sub-micron emulsion.

The process may further comprise the step of cooling the oil in water sub-micron emulsion to, for example, ambient temperature.

According to another aspect, the present disclosure provides a process for the preparation of an oil in water sub-micron emulsion composition, comprising at least one active pharmaceutical ingredient, water, at least one oil, at least one hydrophilic surfactant having an HLB greater than about 10 and optionally at least one hydroxyl containing organic solvent, the process comprising:
(a) heating a mixture of at least one oil, at least one hydrophilic surfactant having an HLB greater than about 10, optionally at least one hydroxyl containing organic solvent and at least one active pharmaceutical ingredient to a temperature above 25° C.;
(b) heating water to a temperature above 25° C.;
(c) combining the heated water with the heated oil mixture;
(d) heating the mixture of step c) to a temperature above the cloud point of the hydrophilic surfactant so as to form a bicontinuous emulsion;
(e) cooling the bicontinuous emulsion to form an oil in water sub-micron emulsion.

The process may further comprise the step of cooling the oil in water sub-micron emulsion composition to, for example, ambient temperature.

According to another aspect, the present disclosure provides a process for the preparation of an oil in water sub-micron emulsion, comprising water, at least one oil, at least one hydrophilic surfactant having an HLB greater than about 10 and optionally at least one hydroxyl containing organic solvent, the process comprising:
(a) admixing at least one oil, at least one hydrophilic surfactant having an HLB greater than about 10 and water;
(b) heating the mixture formed in a) to a temperature above 25° C.;
(c) optionally adding at least one hydroxyl containing organic solvent;
(d) heating the mixture of step c) to a temperature above the cloud point of the hydrophilic surfactant so as to form a bicontinuous emulsion; and
(e) cooling the bicontinuous emulsion to form an oil in water sub-micron emulsion.

The at least one hydroxyl containing solvent may be at a temperature at or above ambient when added in step (c).

According to another aspect, the present disclosure provides a process for the preparation of an oil in water sub-micron emulsion composition, comprising at least one active pharmaceutical ingredient, water, at least one oil, at least one hydrophilic surfactant having an HLB greater than about 10 and optionally at least one hydroxyl containing organic solvent, the process comprising:
(a) admixing at least one oil, at least one hydrophilic surfactant having an HLB greater than about 10 and water;
(b) heating the mixture formed in a) to a temperature above 25° C.;
(c) adding at least one active pharmaceutical ingredient optionally dissolved in at least one hydroxyl containing organic solvent to the heated mixture;
(d) heating the mixture formed in c) to a temperature above the cloud point of the hydrophilic surfactant so as to form a bicontinuous emulsion;
(e) cooling the bicontinuous emulsion to form an oil in water sub-micron emulsion.

The at least one hydroxyl containing solvent may be at a temperature at or above ambient when added in step (c).

In any one or more of the herein disclosed aspects the at least one active pharmaceutical ingredient is soluble in the at least one hydroxyl containing organic solvent.

In any one or more of the herein disclosed aspects the at least one active pharmaceutical ingredient is insoluble or sparingly soluble in water.

In one embodiment the at least one active pharmaceutical ingredient may be initially dissolved in the at least one hydroxyl containing organic solvent prior to combining with the at least one oil and at least one surfactant.

According to another aspect, the present disclosure provides a process for the preparation of an oil in water sub-micron emulsion aerosol foam, comprising at least one oil, at least one hydrophilic surfactant having an HLB greater than about 10, optionally at least one hydroxyl containing organic solvent and water, the process comprising:
(a) heating a mixture of at least one oil, at least one hydrophilic surfactant having an HLB greater than about 10 and optionally at least one hydroxyl containing organic solvent to a temperature above 25° C.;
(b) heating water to a temperature above 25° C.;
(c) combining the heated water with the heated oil mixture;
(d) heating the mixture of step c) to a temperature above the cloud point of the hydrophilic surfactant so as to form a bicontinuous emulsion;
(e) cooling the bicontinuous emulsion to form an oil in water sub-micron emulsion; and
(f) actuating a sample of the sub-micron emulsion with a propellant to form an oil in water sub-micron emulsion aerosol foam.

According to another aspect, the present disclosure provides a process for the preparation of an oil in water sub-micron emulsion aerosol foam composition, comprising at least one active pharmaceutical ingredient, at least one oil, at least one hydrophilic surfactant having an HLB greater than about 10, optionally at least one hydroxyl containing organic solvent and water, the process comprising:
(a) heating a mixture of at least one oil, at least one hydrophilic surfactant having an HLB greater than about 10, optionally at least one hydroxyl containing organic solvent and at least one active pharmaceutical ingredient to a temperature above 25° C.;
(b) heating water to a temperature above 25° C.;
(c) combining the heated water with the heated oil mixture;
(d) heating the mixture of step (c) to a temperature above the cloud point of the hydrophilic surfactant so as to form a bicontinuous emulsion;
(e) cooling the bicontinuous emulsion to form an oil in water sub-micron emulsion; and
(f) actuating a sample of the sub-micron emulsion composition with a propellant to form an oil in water sub-micron emulsion aerosol foam composition.

In one embodiment the at least one active pharmaceutical ingredient may be initially dissolved in the at least one hydroxyl containing organic solvent prior to combining with the at least one oil and at least one surfactant.

In any of the hereinbefore disclosed processes the oil mixture and the water are both heated, to a temperature between about 60 and about 80° C., preferably between about 70 and about 80° C.

In any of the hereinbefore disclosed processes the cloud point may be between about 75 and about 85° C.

In any of the hereinbefore disclosed processes a further step or steps may be performed after cooling the oil in water emulsion. These steps may be performed at ambient temperature or above ambient temperature.

In one embodiment, one or more excipients, which do not substantially interfere with the oil in water emulsion stability, may be added after cooling the emulsion.

In one embodiment the excipient is a humectant, such as glycerine.

According to another aspect, the present disclosure provides a product produced by any one of the hereinbefore disclosed processes.

According to a further aspect, the present disclosure provides a method of treating a disorder or condition, comprising administering to the skin of a subject requiring such treatment an effective amount of a composition of the present disclosure.

According to yet another aspect, the present disclosure relates to the use of the compositions described herein for the preparation of a medicament for the treatment of a disease, disorder or condition.

In some embodiments the oil in water sub-micron emulsion comprises:
(a) up to about 10% by weight of at least one oil;
(b) up to about 15% by weight of at least one hydrophilic surfactant having an HLB greater than about 10;
(c) optionally up to about 20% by weight of at least one hydroxyl containing organic solvent; and
(d) between about 60% and about 90% water.

In some embodiments the oil in water sub-micron emulsion comprises:
(a) between about 1% and about 10% by weight of at least one oil;
(b) between about 1% and about 15% by weight of at least one hydrophilic surfactant having an HLB greater than about 10;
(c) optionally between about 1% and about 20% by weight of at least one hydroxyl containing organic solvent; and
(d) between about 60% and about 90% water.

In some embodiments, the at least one active pharmaceutical ingredient is insoluble or sparingly soluble in water.

In some embodiments the intensity weighted mean diameter of the oil droplets in the sub-micron emulsion or submicron emulsion composition is less than 500 nm, or less than 400 nm, or less than 300 nm, or less than 200 nm, or less than 100 nm. Preferably the mean diameter is less than 200 nm, more preferably less than 100 nm.

In some embodiments 50%, or 60%, or 70%, or 80%, or 90%, of the oil droplets in the sub-micron emulsion or sub-micron emulsion composition have an intensity weighted mean diameter less than 300 nm, or less than 280 nm, or less than 260 nm, or less than 240 nm.

In some embodiments 99% of the oil droplets in the sub-micron emulsion or sub-micron emulsion composition have an intensity weighted mean diameter less than 300 nm, or less than 280 nm, or less than 260 nm, or less than 240 nm.

In some embodiments 50%, or 60%, or 70%, or 80%, or 90% of the oil droplets in the sub-micron emulsion or sub-micron emulsion composition have an intensity weighted mean diameter less than 300 nm, or less than 280 nm, or less than 260 nm, or less than 240 nm, after storage at 5° C. for four weeks.

In some embodiments 99% of the oil droplets in the sub-micron emulsion or sub-micron emulsion composition have an intensity weighted mean diameter less than 300 nm, or less than 280 nm, or less than 260 nm, or less than 240 nm, after storage at 5° C. for four weeks.

In some embodiments 50%, or 60%, or 70%, or 80%, or 90% of the oil droplets in the sub-micron emulsion or sub-micron emulsion composition have an intensity weighted mean diameter less than 300 nm, or less than 280 nm, or less than 260 nm, or less than 240 nm, after storage at 25° C. for four weeks.

In some embodiments 99% of the oil droplets in the sub-micron emulsion or sub-micron emulsion composition have an intensity weighted mean diameter less than 300 nm, or less than 280 nm, or less than 260 nm, or less than 240 nm, after storage at 25° C. for four weeks.

In some embodiments 50%, or 60%, or 70%, or 80%, or 90% of the oil droplets in the sub-micron emulsion or sub-micron emulsion composition have an intensity weighted mean diameter less than 500 nm or less than 400 nm, after storage at 40° C. for four weeks.

In some embodiments 99% of the oil droplets in the sub-micron emulsion or sub-micron emulsion composition have an intensity weighted mean diameter less than 500 nm or less than 400 nm, after storage at 40° C. for four weeks.

In some embodiments the amount of at least one active pharmaceutical ingredient in the sub-micron emulsion composition decreases by less than 10 wt. %, or less than 8 wt. %, or less than 6 wt. %, or less than 4 wt. %, or less than 2 wt. %, when the composition is stored at 40° C. for eight weeks.

In some embodiments the amount of at least one active pharmaceutical ingredient in the sub-micron emulsion composition decreases by less than 10 wt. %, or less than 8 wt. %, or less than 6 wt. %, or less than 4 wt. %, or less than 2 wt. %, when the composition is stored at 40° C. for 12 weeks, or 16 weeks, or 20 weeks, or 24 weeks, or 28 weeks, or 32 weeks or 36 weeks or greater.

In some embodiments the amount of at least one active pharmaceutical ingredient in the sub-micron emulsion composition decreases by less than 10 wt. %, or less than 8 wt. %, or less than 6 wt. %, or less than 4 wt. %, or less than 2 wt. %, when the composition is stored at 25° C. for 12 weeks, or 16 weeks, or 20 weeks, or 24 weeks, or 28 weeks, or 32 weeks or 36 weeks or greater.

In some embodiments the amount of at least one active pharmaceutical ingredient in the sub-micron emulsion composition decreases by less than 10 wt. %, or less than 8 wt. %, or less than 6 wt. %, or less than 4 wt. %, or less than 2 wt. %, when the composition is stored at 25° C. for up to 2 years.

In some embodiments the at least one hydroxyl containing organic solvent is a water miscible organic solvent selected from the group consisting of an alcohol, a glycol, a polyol, and mixtures thereof.

The emulsion or emulsion composition may comprise one or more hydrophilic surfactants. The surfactant may be an anionic surfactant, a cationic surfactant, a non-ionic surfactant, an amphoteric surfactant, or mixtures thereof.

In some embodiments the surfactant is a non-ionic surfactant.

In some embodiments the emulsion or emulsion composition comprises a single surfactant.

In some embodiments the emulsion or emulsion composition is free, or substantially free, of lipophilic surfactants.

Alternatively the emulsion or emulsion composition comprises more than one surfactant wherein the HLB values of the surfactants are within 30% of each other, preferably within 20% of each other.

The emulsion or emulsion composition may comprise up to 15% by weight of one or more surfactants based on the total weight of the emulsion or emulsion composition, or up to 10% by weight, or up to 7.5% by weight. The emulsion or emulsion composition may comprise about 2% to about 15% by weight surfactant, or about 5 to about 10% by weight surfactant, or about 6 to about 9% by weight, based on the total weight of the emulsion or emulsion composition.

In some embodiments the emulsion or emulsion composition may have a pH less than or equal to 7.0, or less than or equal to 6.5, or less than or equal to 6.0, or less than or equal to 5.5, or less than or equal to 5.0.

In some embodiments the emulsion or emulsion composition may have a pH in the range from about 7.0 to about 3.0, or from about 7.0 to about 3.5, or from about 7.0 to about 4.0, or from about 6.0 to about 3.0, or from about 6.0 to about 4.0, or from about 5.0 to about 3.0, or from about 5.0 to about 3.5, or from about 5.0 to about 4.0.

In other embodiments the emulsion or emulsion composition may have a pH greater than about 7.0, or greater than about 7.5, or greater than about 8.0, or greater than about 8.5, or greater than about 9.0.

In other embodiments the emulsion or emulsion composition may have a pH in the range from about 7.0 to about 11.0, or from about 7.0 to about 10.0, or from about 7.0 to about 9.0.

In some embodiments the emulsion or emulsion composition may have a pH that changes by less than 0.5 pH units, or less than 0.4 pH units, or less than 0.3 pH units, when the emulsion or emulsion composition is stored for a period of one year at a temperature of 40° C.

In some embodiments the combined amount of surfactant and oil in the emulsion or emulsion composition may be less than 25% by weight, or less than 20% by weight, or less than 15% by weight.

In some embodiments the emulsion or the emulsion composition may have a dynamic viscosity of less than <100 cps.

The sub-micron emulsions or emulsion compositions may comprise one or more acceptable carriers and excipients, including preservatives, anti-oxidants, anti-inflammatories, emollients, moisturisers, buffers, humectants, solubilisers, fragrances, colourants, viscosity modifying agents and essential oils, selected to facilitate and/or enhance application, user experience and/or efficacy. Other agents may be contemplated. The further components are preferably miscible and compatible and do not detract from the function of the pharmaceutically active ingredient or detract from the stability and function of the emulsion.

### Advantages of the Present Sub-Micron Emulsions

The sub-micron emulsions of the present disclosure may possess one or more of the following advantages compared to prior art sub-micron emulsions.
only hydrophilic surfactants are required to produce a stable sub-micron emulsion
only a single surfactant is required to form a stable sub-micron emulsion
the sub-micron emulsions are stable over long time periods
the sub-micron emulsions may be foamed to provide stable foams.

### BRIEF DESCRIPTION OF THE DRAWINGS

FIG. 1 is a flow diagram of a prior art process for preparing a sub-micron emulsion composition which utilizes both hydrophilic and lipophilic surfactants.

FIG. 2 is a flow diagram of a process for preparing a sub-micron emulsion composition according to an embodiment of the present disclosure where a thermolabile active pharmaceutical ingredient is added after the sub-micron emulsion if formed and cooled.

FIG. 3 is a flow diagram of a process for preparing a sub-micron emulsion composition according to an embodiment of the present disclosure where a thermostable active pharmaceutical ingredient is added prior to emulsion formation.

FIG. 4 is a flow diagram of a process for preparing a sub-micron emulsion composition according to an embodiment of the present disclosure.

FIG. 5 is a graph of conductivity vs temperature of a sub-micron emulsion composition according to an embodiment of the present disclosure.

FIG. 6 shows the particle size distribution of a sub-micron emulsion composition according to an embodiment of the present disclosure.

FIG. 7 shows the particle size distribution of a sub-micron emulsion composition according to an embodiment of the present disclosure after storage at 5° C. for four weeks.

FIG. 8 shows the particle size distribution of a sub-micron emulsion composition according to an embodiment of the present disclosure after storage at 25° C. for four weeks.

FIG. 9 shows the particle size distribution of a sub-micron emulsion composition according to an embodiment of the present disclosure after storage at 40° C. for four weeks.

FIG. 10 is a photograph of a sub-micron emulsion foam composition according to an embodiment of the present disclosure.

### DETAILED DESCRIPTION OF THE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure.

Although any compositions, processes and materials similar or equivalent to those described herein can also be used in the practice or testing of the present disclosure, the preferred compositions, processes and materials are now described.

It must also be noted that, as used in the specification and the appended claims, the singular forms ‘a’, ‘an’ and ‘the’ include plural referents unless otherwise specified. Thus, for example, reference to ‘oil’ may include more than one oil, and the like.

Throughout this specification, use of the terms ‘comprises’ or ‘comprising’ or grammatical variations thereon shall be taken to specify the presence of stated features, integers, steps or components but does not preclude the presence or addition of one or more other features, integers, steps, components or groups thereof not specifically mentioned.

Unless specifically stated or obvious from context, as used herein, the term ‘about’ is understood as within a range of normal tolerance in the art, for example within two standard deviations of the mean. ‘About’ can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term ‘about’.

Any processes provided herein can be combined with one or more of any of the other processes provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Definitions

As used herein, the terms ‘administering’ and ‘administered’ refer to any method which delivers the present compositions to a subject in such a manner as to provide a therapeutic effect.

As used herein, the term ‘derivative(s) thereof’ refers to prodrugs, solvates, hydrates, esters and acids of the active pharmaceutical ingredient.

As used herein, the phrase an ‘effective amount’ of an active agent or ingredient, or active pharmaceutical ingredient or ingredient, which are synonymous herein, refers to an amount of the active pharmaceutical ingredient sufficient to have a therapeutic effect upon administration. An effective amount of the active agent may, will, or is expected to cause relief of symptoms. Effective amounts of the active agent will vary with the particular disease or diseases being treated, the severity of the disease, the duration of the treatment, and the specific components of the composition being used.

As used herein, the droplet size or particle size of an emulsion refers to the intensity weighted mean diameter as measured by dynamic light scattering.

As used herein, a ‘submicron emulsion’ refers to a mixture of two or more immiscible liquids wherein the droplet or particle diameter of the dispersed phase (i.e. oil in the case of an oil in water emulsion) is in the range from about 20 nm to about 1000 nm expressed as an intensity weighted distribution.

As used herein, the term ‘bicontinuous emulsion’ is an emulsion composition wherein oil in water and water in oil emulsions coexist as a mixture. By ‘coexist as a mixture’ is meant that the microstructure of the emulsion fluid is such that regions of oil in water intermingle with regions of water in oil. A bicontinuous emulsion exhibits regions of water continuity and regions of oil continuity. A bicontinuous emulsion is by character a micro-heterogeneous biphasic fluid.

As used herein, the term ‘cloud point’ refers to the temperature above which a non-ionic surfactant or wax loses some of its water solubility and becomes ineffective as a surfactant.

As used herein, the term ‘phase inversion temperature’ refers to a temperature where an oil in water emulsion inverts to a water in oil emulsion or vice versa.

As used herein, a ‘pH adjusting agent’ refers to a specific pH adjusting agent or agents, including but not limited to, a buffer, a base or an acid, salts thereof and mixtures thereof, added to a composition.

As used herein, the phrase ‘pharmaceutically acceptable salts’ refers to salts that are pharmaceutically acceptable and that possess the desired pharmacological activity of the parent compound. Such salts include: (1) acid addition salts, formed with acids such as, for example, acetic acid, benzoic acid, citric acid, gluconic acid, glutamic acid, glutaric acid, glycolic acid, hydrochloric acid, lactic acid, maleic acid, malic acid, malonic acid, mandelic acid, phosphoric acid, propionic acid, sorbic acid, succinic acid, sulfuric acid, tartaric acid, naturally and synthetically derived amino acids, and mixtures thereof; or (2) salts formed when an acidic proton present in the parent compound is either (i) replaced by a metal ion e.g. an alkali metal ion, an alkaline earth metal ion, or an aluminium ion; or (ii) protonates an organic base such as, for example, ethanolamine, diethanolamine, triethanolamine, tromethamine and N-methylglucamine.

As used herein, a ‘subject’, ‘individual’ or ‘patient’ refers to any subject, particularly a human, for whom therapy is desired.

As used herein, a ‘treatment’ or ‘treating’ of a disease, disorder or condition encompasses alleviation of at least one symptom thereof, a reduction in the severity thereof, or the delay, prevention or inhibition of the progression thereof. Treatment need not mean that the disease, disorder or condition is totally cured. A useful composition herein need only to reduce the severity of a disease, disorder or condition, reduce the severity of symptoms associated therewith, provide improvement to a patient's quality of life, or delay, prevent or inhibit the onset of a disease, disorder or condition.

As used herein, the term ‘substantially free’ of a specified component refers to a composition with less than about 1% of the specified component.

The present disclosure is directed to an oil in water sub-micron emulsion or sub-micron emulsion composition, said emulsion having a water phase and an oil phase. In an embodiment, the present disclosure provides low oil and low surfactant content, oil in water emulsion aerosol foam compositions.

Thus, according to one embodiment the present disclosure provides an oil in water sub-micron emulsion, said emulsion comprising an oil phase dispersed throughout a water phase, said emulsion comprising:
(a) between about 1% and about 10% by weight of at least one oil;
(b) between about 1% and about 15% by weight of at least one hydrophilic surfactant having an HLB value greater than about 10;
(c) optionally between about 1% and about 20% by weight of at least one hydroxyl containing organic solvent; and
(d) the balance as water;

wherein the at least one surfactant is miscible both with water and the at least one hydroxyl containing organic solvent.

According to another embodiment the present disclosure provides an oil in water sub-micron emulsion composition, said composition comprising:
(a) at least one active pharmaceutical ingredient; and
(b) an oil in water sub-micron emulsion comprising an oil phase dispersed throughout a water phase, said oil in water sub-micron emulsion comprising:
(i) between about 1% and about 10% by weight of at least one oil;
(ii) between about 1% and about 15% by weight of at least one hydrophilic surfactant having an HLB greater than about 10;
(iii) optionally between about 1% and about 20% by weight of at least one hydroxyl containing organic solvent; and
(iv) the balance as water;

wherein the at least one surfactant is miscible both with water and the at least one hydroxyl containing organic solvent.

According to another embodiment the present disclosure provides an oil in water sub-micron emulsion aerosol foam comprising an oil phase dispersed throughout a water phase, said foam comprising:
(a) between about 1% and about 10% by weight of at least one oil;
(b) between about 1% and about 15% by weight of at least one hydrophilic surfactant having an HLB greater than about 10;
(c) optionally between about 1% and about 20% by weight of at least one hydroxyl containing organic solvent; and
(d) water;

wherein the at least one surfactant is miscible both with water and the at least one hydroxyl containing organic solvent.

According to another embodiment the present disclosure provides an oil in water sub-micron emulsion aerosol foam composition said, the composition comprising:
(a) at least one active pharmaceutical ingredient; and
(b) an oil in water sub-micron emulsion foam comprising an oil phase dispersed throughout a water phase, said oil in water sub-micron emulsion foam composition comprising:
(i) between about 1% and about 10% by weight of at least one oil;
(ii) between about 1% and about 15% by weight of at least one hydrophilic surfactant having an HLB value greater than about 10;
(iii) optionally between about 1% and about 20% by weight of at least one hydroxyl containing organic solvent; and
(iv) water;

wherein the at least one surfactant is miscible both with water and the at least one hydroxyl containing organic solvent.

Together, the oil and optional hydroxyl containing organic solvent comprise the oil phase of the composition, along with any oil miscible excipients.

According to an embodiment, the intensity weighted mean particle or droplet diameter of the oil phase is about 100 nm.

Oil Component

Suitably, the oil is present in the composition in an amount from about 1% to about 10% by weight. In another embodiment the oil is present in an amount from about 3% to about 9% by weight, such as about 3%, 4%, 5%, 6%, 7% or 8% by weight.

The oil is in the internal phase of the oil in water emulsion system. In an embodiment, the oil is a hydrocarbon. Suitably, the hydrocarbon is selected from a linear, branched or cyclic alkane or alkene, or mixtures thereof.

According to a further embodiment, the linear, branched or cyclic alkane or alkene is selected from the group consisting of isoparaffin, dodecene, diethylhexylcyclohexane, eicosane, isododecane, isoeicosane, isohexadecane, longifolene, mineral oil, paraffin, pentahydrosqualene, petrolatum, squalane, squalene, tetradecene, derivatives thereof, and mixtures thereof.

According to an embodiment, the oil is mineral oil. In one embodiment, the mineral oil is present in an amount from about 1% to about 9% by weight. In another embodiment the mineral oil is present in about 3% to about 8% by weight, such as about 3%, 4%, 5%, 6%, 7% or 8% by weight.

In another embodiment, the oil is a vegetable oil. Suitably, the vegetable oil is selected from palm oil, soybean oil, rapeseed oil, sunflower oil, peanut oil, corn oil, olive oil, coconut oil, cottonseed oil, linseed oil, grapeseed oil, hazelnut oil or sesame oil, and mixtures thereof.

Surfactant Component

The present sub-micron emulsions and emulsion compositions comprise a surfactant component. Suitably, the surfactant is present in an amount from about 1% to about 15% by weight. In another embodiment the surfactant is present in an amount from about 2% to about 12% by weight, such as about 2%, 3%, 4%, 5%, 6%, 7%, 8%, 9%, 10%, 11% or 12% by weight.

A surfactant's hydrophilic/lipophilic balance (HLB) describes the surfactant's affinity toward water or oil. The HLB scale ranges from 1 (totally lipophilic) to 20 (totally hydrophilic), with 10 representing an equal balance of both characteristics. The HLB of a blend of two surfactants equals the weight fraction of surfactant A times its HLB value plus the weight fraction of surfactant B times its HLB value (weighted average).

In one embodiment the surfactant component comprises a single hydrophilic surfactant, and in another embodiment, the surfactant component comprises more than one hydrophilic surfactant and the weighted average of their HLB values is greater than about 10, or between about 10 and about 24.

In another embodiment, the surfactant component consists of non-ionic surfactants.

Suitable non-ionic surfactants include but are not limited to ethoxylated fatty alcohol ethers, PEG derivatives, ethoxylated fatty acids, propylene glycol esters, fatty alcohols, glycerol esters and derivatives, polymeric ethers and sorbitan esters, and mixtures thereof.

In one embodiment the hydrophilic ethoxylated fatty alcohol ether is selected from the group consisting of steareth-10, steareth-20, ceteareth-10, ceteareth-12, ceteareth-15, ceteareth-20, ceteareth-21, ceteareth-22, ceteareth-25, ceteareth-30, ceteareth-31, ceteareth-32, ceteareth-33, ceteareth-6, laureth-5, laureth-9, laureth-10, laureth-12, laureth-15, laureth-20, laureth-21, laureth-22, laureth-23, nonoxynol-9, oleth-10 and oleth-20.

In another embodiment the hydrophilic ethoxylated fatty alcohol ether is oleth-10. In one embodiment the oleth-10 is present in the composition in an amount from about 1% to about 15% by weight. In another embodiment the oleth-10 is present in an amount from about 2% to about 12% by weight, such as about 2%, 3%, 4%, 5%, 6%, 7%, 8%, 9%, 10%, 11% or 12% by weight. In a preferred embodiment the Oleth-10 is present in an amount between 5 and 10% by weight.

Exemplary PEG derivatives include PEG-25 hydrogenated castor oil, PEG-30 castor oil, PEG-31 castor oil, PEG-32 castor oil, PEG-33 castor oil, PEG-34 castor oil, PEG-35 castor oil, PEG-40 hydrogenated castor oil, PEG-50 castor oil and PEG-60 hydrogenated castor oil.

Exemplary ethoxylated fatty acids include, PEG-6 oleate, PEG-10 oleate, PEG-6 stearate, PEG-8 stearate and PEG-9 stearate, PEG-20 stearate, PEG-40 stearate, PEG-41 stearate, PEG-42 stearate, PEG-43 stearate, PEG-44 stearate, PEG-45 stearate, PEG-46 stearate, PEG-47 stearate, PEG-48 stearate, PEG-49 stearate, PEG-50 stearate and PEG-100 stearate.

Exemplary propylene glycol esters include propylene glycol palmitate and propylene glycol stearate.

Exemplary fatty alcohols include cetyl alcohol and stearyl alcohol.

Exemplary glyceryl esters and derivatives include glyceryl behenate, glyceryl dibehenate, glyceryl dioleate, glyceryl distearate, glyceryl linoleate, glyceryl oleate, glyceryl stearate, PEG-23 glyceryl cocoate, PEG-6 caprylic/capric glycerides, PEG-7 glyceryl cocoate, polyglyceryl-10 diisostearate, polyglyceryl-2 diisostearate, polyglyceryl-3 diisostearate and polyglyceryl-6 diisostearate.

Exemplary polymeric ethers include poloxamer 124, poloxamer 182, poloxamer 184, poloxamer 188, poloxamer 237, poloxamer 331, poloxamer 338 and poloxamer 407.

Exemplary sorbitan derivatives include polysorbate 20, polysorbate 40, polysorbate 60, polysorbate 80, sorbitan laurate, sorbitan oleate, sorbitan palmitate, sorbitan sesquioleate, sorbitan stearate and sorbitan trioleate.

In one embodiment, the surfactant component comprises one or more hydrophilic non-ionic surfactants and is substantially free, or free of lipophilic surfactant.

In one embodiment, the surfactant component comprises a hydrophilic ethoxylated fatty alcohol ether. In another embodiment, the surfactant component comprises a hydrophilic ethoxylated fatty alcohol ether and is substantially free, or free of lipophilic surfactant.

In one embodiment the surfactant has an HLB between about 10 and about 18, or between about 11 and about 17, or between about 11 and about 16, or between about 12 and about 14. Preferably the surfactant HLB is between about 12 and about 14.

Active Pharmaceutical Ingredient

Suitably, the pharmaceutically acceptable active agent for use in the compositions herein may be selected from corticosteroids, antihistamines, antibacterial agents, antifungal agents, antiviral agents, antiseptics, sunscreens, anaesthetics, analgesics, and skin conditioning agents, salts thereof, derivatives thereof and mixtures thereof. In one embodiment, the compositions may comprise more than one active pharmaceutical ingredient, salt or derivative thereof. Suitable concentration ranges for the active pharmaceutical ingredient range from about 0.001% to about 30% by weight, depending on the nature of the active agent or combination of active agents. Preferably, the active pharmaceutical ingredient is present in an amount from about 0.01% to about 5% by weight or in an amount from about 0.02% to about 2% by weight.

Suitable corticosteroids include, but are not limited to, alclometasone dipropionate, amcinonide, beclomethasone dipropionate, betamethasone benzoate, betamethasone dipropionate, betamethasone valerate, budesonide, clobetasol propionate, clobetasone butyrate, cortisone acetate, desonide, desoximetasone, diflorasone diacetate, diflucortolone valerate, fluclorolone acetonide, flumethasone pivalate, fluocinolone acetonide, fluocinonide, fluocortin butyl, fluocortolone, fluprednidene acetate, flurandrenolide, flurandrenolone, fluticasone propionate, halcinonide, halobetasol propionate, hydrocortisone, hydrocortisone acetate, hydrocortisone butyrate, hydrocortisone propionate, hydrocortisone valerate, methylprednisolone acetate, mometasone furoate, pramoxine hydrochloride, prednisone acetate, prednisone valerate, triamcinolone acetonide, prednicarbate, salts thereof, derivatives thereof, and mixtures thereof.

Suitable antihistamines include, but are not limited to, cetirizine, vapitadine, diphenhydramine, triprolidine, pyrilamine, chlorcyclizine, promethazine, carbinoxamine, tripelennamine, brompheniramine, hydroxyzine, terfenadine, chlorpheniramine, salts thereof, derivatives thereof, and mixtures thereof. A particularly preferred corticosteroid is hydrocortisone butyrate.

Suitable antibacterial agents include, but are not limited to, gentamicin, neomycin, streptomycin, cefpodoxime proxetil, clindamycin, lincomycin, erythromycin, bacitracin, gramicidin(s), vancomycin, doxycycline, minocycline, oxytetracycline, tetracycline, fosfomycin, fusidic acid, mupirocin, sulfacetamide, metronidazole and dapsone, salts thereof, derivatives thereof, and mixtures thereof.

Suitable antifungal agents include, but are not limited to, those selected from the group consisting of echinocandins such as anidulafunin, caspofungin and micafungin; polyenes such as amphotericin B, candicidin, filipin, fungichromin, hachimycin, hamycin, lucensomycin, mepartricin, natamycin, nystatin, pecilocin, perimycin; allylamines such as butenafine, naftifine and terbinafine; imidazoles such as bifonazole, butoconazole, chlormidazole, cloconazole, clotrimazole, econazole, enilconazole, fenticonazole, flutrimazole, isoconazole, ketoconazole, lanoconazole, miconazole, neticonazole, omoconazole, oxiconazole nitrate, sertaconazole, sulconazole and tioconazole; thiocarbamates such as liranaftate, tolciclate, tolindate and tolnafate; triazoles such as albaconazole, fluconazole, itraconazole, posaconazole, ravuconazole, saperconazole, terconazole and voriconazole; and other antifungal agents such as acrisorcin, amorolfine, biphenamine, bromosalicylchloranilide, buclosamide, calcium propionate, chlorphenesin, ciclopirox, cloxyquin, coparaffinate, exalamide, flucytosine, haloprogin, hexetidine, loflucarban, nifuratel, potassium iodide, propionic acid, pyrithione, salicylanilide, sodium propionate, sulbentine, tenonitrozole, triacetin, undecylenic acid, zinc propionate, griseofulvin, oligomycins, pyrrolnitrin, siccanin, viridian, salts thereof, derivatives thereof, and mixtures thereof.

Suitable antivirals include, but are not limited to, acyclovir, desciclovir, carbovir, famciclovir, foscarnet sodium, ganciclovir, interferons, penciclovir, valaciclovir, salts thereof, derivatives thereof, and mixtures thereof.

Suitable antiseptics include, but are not limited to, hydrogen peroxide, chlorhexidine, cetrimide, povidone iodine, triclosan, salts thereof, derivatives thereof, and mixtures thereof.

Suitable anaesthetics and analgesics include, but are not limited to, benzocaine, lidocaine, prilocaine and choline salicylate, salts thereof, derivatives thereof, and mixtures thereof.

Suitable skin-conditioning agents include, but are not limited to, hydrocarbon oils and waxes, silicones, fatty acid derivatives, cholesterol, di- and tri-glycerides, vegetable oils, acetoglyceride esters, alkyl esters, alkenyl esters, lanolin, milk tri-glycerides, wax esters, beeswax, sterols, phospholipids, derivatives thereof, and mixtures thereof.

Water Phase

The emulsions and emulsion compositions of the present disclosure also comprise water. Water forms the continuous phase of the emulsion system. In an embodiment, the emulsions and emulsion compositions comprise water in an amount from about 55% to about 95% by weight. In another embodiment the water is present in an amount from about 60% to about 90% by weight. In another embodiment the water is present in an amount from about 65% to about 85% by weight.

Hydroxyl Containing Organic Solvent

The present emulsions and emulsion compositions optionally comprise a hydroxyl containing organic solvent to, when necessary or desirable, facilitate solubilisation of the active pharmaceutical ingredient.

According to an embodiment, the hydroxyl containing organic solvent is present in an amount from about 1% to about 20% by weight. According to a further embodiment, the hydroxyl containing organic solvent is present in an amount of about 10% by weight.

Suitable hydroxyl containing organic solvents include, but are not limited to, alcohols, including amyl alcohol, benzyl alcohol, cyclohexanedimethanol, diacetone alcohol, ethyl alcohol, hexyl alcohol, isobutyl alcohol, isopropyl alcohol, methyl alcohol, n-butyl alcohol, propyl alcohol, t-butyl alcohol, tetrahydrofurfuryl alcohol; carboxylic acids, including acetic acid; diols, including 1,2-hexanediol, butylene glycol, diethylene glycol, dipropylene glycol, ethyl hexanediol, ethylene glycol, hexylene glycol, pentylene glycol, propylene glycol, tetraethylene glycol, triethylene glycol, tripropylene glycol; and polyols including polyethylene glycol, butanetriol, glycerol and 1,2,6-hexanetriol.

In one embodiment, the hydroxyl containing organic solvent is propylene glycol.

Propellant

The present foams and foam compositions may utilise a propellant in order to produce the foam upon application. The propellant may be any suitable liquefied gas or mixture thereof, such as a hydrocarbon, a chlorofluorocarbon, dimethyl ether, hydrofluorocarbons and a mixture thereof.

Other suitable propellants include compressed gases such as nitrogen, carbon dioxide, nitrous oxide and air. In a preferred embodiment, the propellant is a mixture of hydrocarbons. In a further preferred embodiment, the mixture of hydrocarbons is a mixture of propane, n-butane and isobutane.

The propellant is present in an amount from about 1% to about 20% by weight, or about 3% to about 15% by weight. In one embodiment, the propellant is present in an amount from about 5% to about 10% by weight, such as about 5%, 6%, 7%, 8%, 9% or 10% by weight. The propellant may be introduced into the composition at the time of filling, utilising a pressurised container such as a standard aerosol dispenser.

When the composition is released from the pressurised container, the composition is an aerosol foam (also known as a mousse). According to one embodiment, the aerosol foam is homogeneous. In another embodiment, the aerosol foam breaks easily with shear, such as gentle mechanical action e.g. rubbing or spreading.

In another embodiment the propellant is absent from the composition. According to such an embodiment, the composition may be expelled from its container by mechanical means, such as by a pump action or a squeezing action on the container.

Suitable pressurized containers for use herein include aluminium, tin-plate and glass containers.

In one embodiment, the pressurized container is a one-piece aluminium container in which the inner surface is lined with a chemically inert lining. One suitable inner surface lining for use herein is polyamide-imide (PAM). The container may be fitted with an upright-use or inverted-use valve and a conventional foam spout actuator. Alternatively, the container may be fitted with a metered-dose valve.

Excipients

According to an embodiment, the emulsions or emulsion compositions may further comprise one or more excipients. Non-limiting examples of excipients include diluents, suspending agents, adjuvants, preservatives, colorants, emollients, pH adjusting agents (including buffers), thickeners, humectants, fragrances, stabilisers, chelating agents, anticaking agents, viscosity increasing agents, moisturisers, solubilisers, plasticisers, penetration enhancing agents, film forming agents, antioxidants, wetting agents, foam boosters or any mixture of these components.

In one embodiment, the one or more excipients comprise a preservative, an antioxidant, a pH adjusting agent and a humectant.

Preservative

The present sub-micron emulsions or emulsion compositions may additionally comprise a preservative. The preservative is present in the composition in an amount from about 0.01% to about 3% by weight. In one embodiment the preservative is present in an amount from about 0.1% to about 2% by weight. In another embodiment the preservative is present in an amount of about 1% by weight.

Suitable preservatives include, but are not limited to, benzyl alcohol, diazolidinyl urea, methyl paraben, ethyl paraben, propyl paraben, butyl paraben, phenoxyethanol, sorbic acid and salts thereof such as potassium sorbate, benzoic acid and salts thereof such as sodium benzoate, and mixtures thereof. The composition may also comprise one or more salts of EDTA, for example sodium salts of EDTA, such as Na₂EDTA. These may serve to enhance preservation.

According to an embodiment, the preservative is benzyl alcohol.

Antioxidant

The present sub-micron emulsions or emulsion compositions may further comprise an antioxidant. The antioxidant is present in the composition in an amount from about 0.001% to about 1% by weight. In one embodiment the antioxidant is present from about 0.05% to about 0.5% by weight. In another embodiment the antioxidant is present in an amount of about 0.1% by weight.

Suitable antioxidants include, but are not limited to, butylated hydroxytoluene (BHT), butylated hydroxyanisole, tocopherol, propyl gallate, dl-α-tocopherol, vitamin E TPGS, derivatives thereof, and mixtures thereof. In one embodiment, the antioxidant is BHT.

pH Adjusting Agent

The present sub-micron emulsion compositions may further comprise a pH adjusting agent to aid in stabilizing the active pharmaceutical ingredient. According to an embodiment, the pH adjusting agent is present in an amount from about 0.01% to about 10% by weight. In one embodiment, the pH adjusting agent is a base. Suitable pH adjusting bases include but are not limited to amines, bicarbonates, carbonates and hydroxides (such as alkali or alkaline earth metal hydroxides, as well as transition metal hydroxides). The pH adjusting agent may also be an acid, an acid salt, or mixtures thereof. The pH adjusting agent may also be a buffer. Suitable buffers include, but are not limited to citrate/citric acid, acetate/acetic acid, phosphate/phosphoric acid, formate/formic acid, propionate/propionic acid, lactate/lactic acid, carbonate/carbonic acid, ammonium/ammonia, derivatives thereof, and combinations thereof. According to an embodiment, the pH adjusting agent is a citrate/citric acid buffer. According to an embodiment, the citrate/citric acid buffer is present in an amount from about 0.02% to about 2% by weight.

Humectant

The sub-micron emulsions or emulsion compositions of the present disclosure may comprise one or more humectants included to enhance the performance of the composition. Any humectant known in the art for use in pharmaceutical compositions may be used, for example, glycerine.

The composition may comprise up to 30% by weight of one or more humectants, based on the total weight of the composition, or up to about 20% by weight, or up to about 15% by weight. The humectant may be present in an amount of about 1 to about 20% by weight, or about 2 to about 15%, or about 3 to about 10%, based on the total weight of the composition.

Process of Preparation

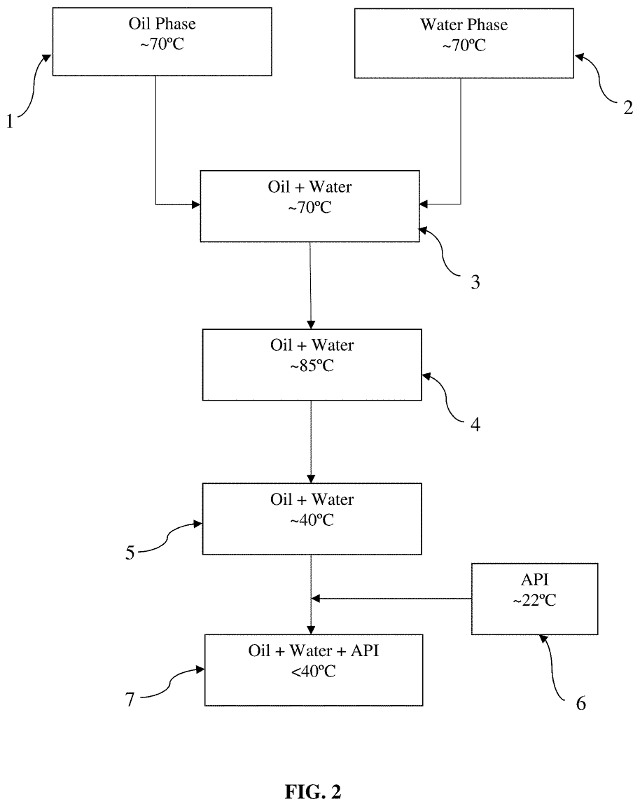

Preferred embodiments of processes for preparing the sub-micron emulsion compositions according to the present disclosure are outlined in FIGS. 2-4.

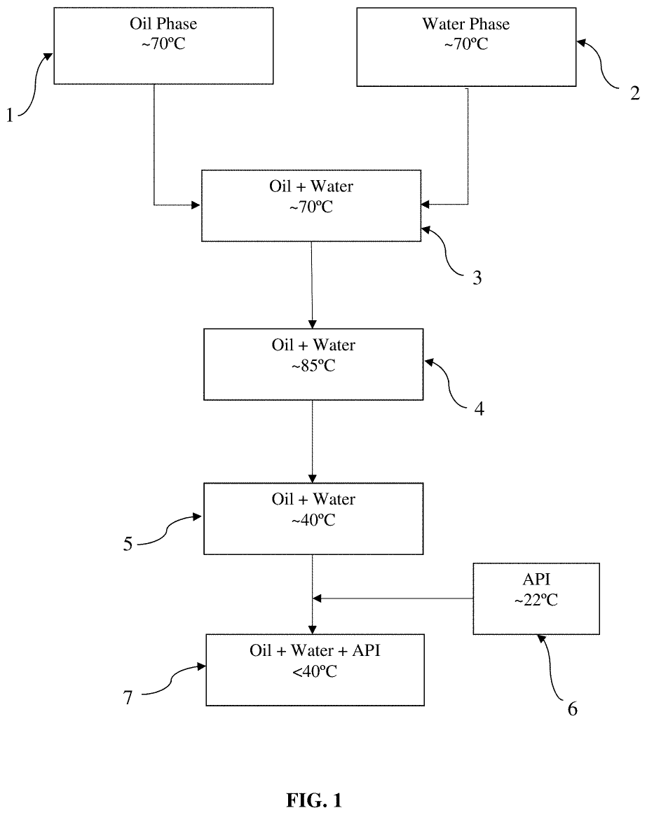

FIG. 1 is a flow chart of a prior art process for producing a sub-micron emulsion composition in which both lipophilic and hydrophilic surfactants are utilized and the active pharmaceutical ingredient is added after emulsion formation. Oil phase (1) is prepared including mineral oil, hydrophilic surfactant Ceteareth-20 (HLB=15.2) and lipophilic surfactant Oleth-5 (HLB=9.4). The oil phase may contain other components such as antioxidant BHT and preservative benzyl alcohol. The oil phase is heated to a temperature of about 70° C. Water phase (2) is prepared including water and optional further components, such as buffer citric acid/sodium citrate, preservative disodium EDTA and humectant glycerine. The water phase is heated to a temperature of about 70° C. The heated water phase is added to the heated oil phase to provide oil and water mixture (3). This mixture is further heated to about 85° C. at which point the cloud point is observed. The resulting emulsion (4) is then cooled to about 40° C. (5) at which point a solution of active pharmaceutical ingredient (API; hydrocortisone butyrate) in propylene glycol at about 22° C. (6) is added to yield sub-micron emulsion composition (7).

The resulting sub-micron emulsion composition has an intensity weighted mean oil droplet diameter of about 56 nm. Similar results are obtained if the glycerine is added after emulsion formation rather than with the water phase.

FIG. 2 is a flow chart of a process for producing a sub-micron emulsion composition according to one embodiment of the present disclosure in which a single hydrophilic surfactant is utilized and the active pharmaceutical ingredient is added after emulsion formation. Oil phase (1) is prepared including mineral oil and hydrophilic surfactant Oleth-10 (HLB=12.4). The oil phase may contain other components such as antioxidant BHT and preservative benzyl alcohol. The oil phase is heated to a temperature of about 70° C. Water phase (2) is prepared including water and optional further components, such as buffer citric acid/sodium citrate, preservative disodium EDTA and humectant glycerine. The water phase is heated to a temperature of about 70° C. The heated water phase is added to the heated oil phase to provide oil and water mixture (3). This mixture is further heated to about 85° C. at which point the cloud point is observed. The resulting mixture (4) is then cooled to about 40° C. (5) at which point a solution of active pharmaceutical ingredient (API; hydrocortisone butyrate) in propylene glycol at about 22° C. (6) is added to yield a sub-micron emulsion composition (7).

The resulting sub-micron emulsion composition has an intensity weighted mean oil droplet diameter of about 20 nm. Similar results are obtained if the glycerine is added after emulsion formation rather than with the water phase.

Ceteareth-12 (HLB=13.5) may also be used as hydrophilic surfactant.

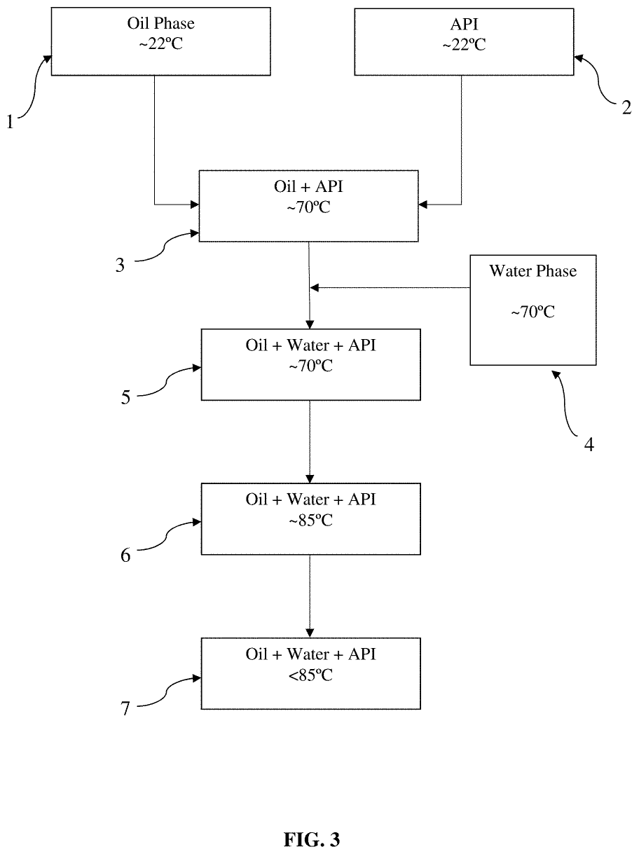

FIG. 3 is a flow chart of a process for producing a sub-micron emulsion composition according to another embodiment of the present disclosure in which a single hydrophilic surfactant is utilized and the active pharmaceutical ingredient is added before emulsion formation. Oil phase (1) is prepared including mineral oil and hydrophilic surfactant Oleth-10 (HLB=12.4). The oil phase may contain other components such as antioxidant BHT and preservative benzyl alcohol. A solution of active pharmaceutical ingredient (API; hydrocortisone butyrate) in propylene glycol at about 22° C. (2) is prepared. The API solution is added to the oil phase and the resulting mixture (3) heated to about 70° C. Water phase (4) is prepared including water and optional further components, such as buffer citric acid/sodium citrate, preservative disodium EDTA and humectant glycerine. The water phase is heated to a temperature of about 70° C. The heated water phase is added to the heated oil phase to provide oil and water mixture (5). This mixture is further heated to about 85° C. at which point the cloud point is observed (6). The mixture (6) is then cooled to yield sub-micron emulsion composition (7).

The resulting sub-micron emulsion composition has an intensity weighted mean oil droplet diameter of about 60 nm. Similar results are obtained if the glycerine is added after emulsion formation rather than with the water phase.

Ceteareth-12 (HLB=13.5) may also be used as hydrophilic surfactant.

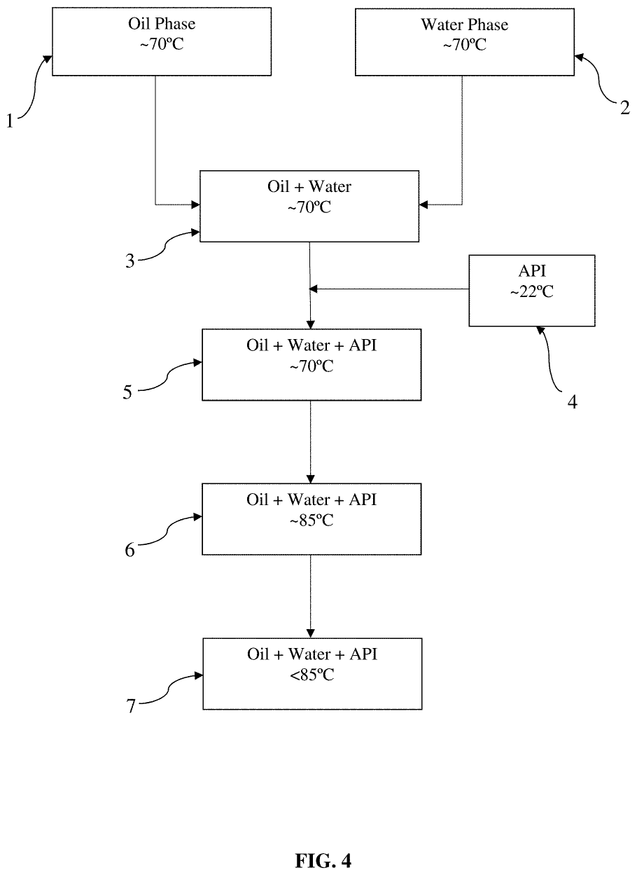

FIG. 4 is a flow chart of a process for producing a sub-micron emulsion composition according to one embodiment of the present disclosure in which a single hydrophilic surfactant is utilized and the active pharmaceutical ingredient is added before emulsion formation. Oil phase (1) is prepared including mineral oil and hydrophilic surfactant Oleth-10 (HLB=12.4). The oil phase may contain other components such as antioxidant BHT and preservative benzyl alcohol. The oil phase is heated to a temperature of about 70° C. Water phase (2) is prepared including water and optional further components, such as buffer citric acid/sodium citrate, preservative disodium EDTA and humectant glycerine. The water phase is heated to a temperature of about 70° C. The heated water phase is added to the heated oil phase to provide oil and water mixture (3). A solution of active pharmaceutical ingredient (API; hydrocortisone butyrate) in propylene glycol at about 22° C. (4) is then added and the resulting mixture (5) further heated to about 85° C. at which point the cloud point is observed. The resulting mixture (6) is then cooled to yield sub-micron emulsion composition (7).

The resulting sub-micron emulsion composition has an intensity weighted mean oil droplet diameter of about 20 nm. Similar results are obtained if the glycerine is added after emulsion formation rather than with the water phase.

Ceteareth-12 (HLB=13.5) may also be used as hydrophilic surfactant.

Methods of Treatment

The emulsion aerosol foam compositions of the present disclosure are cosmetically elegant and suitable for application to the face, hands and other parts of the body including scalp, feet, legs, arms and trunk, for treating a skin disorder or condition. The compositions are useful for application to both large and small skin surfaces. The compositions are also useful for application to hair bearing areas. The compositions are easily spread, non-greasy, non-drying and leave minimal residue on the skin.

The present disclosure provides for a method of treating a skin disease, disorder or condition, comprising administering to the skin of a patient requiring such treatment an effective amount of a composition of the present disclosure.

The present disclosure also relates to the use of the compositions as described herein for the preparation of a medicament for the treatment of a skin disease, disorder or condition.

The present disclosure also relates to a method of treating a skin disease, disorder or condition by administering to the skin of a patient requiring such treatment an effective amount of a composition of the present invention.

Exemplary, non-limiting, skin diseases, disorders or conditions treatable by the present compositions include acne, rosacea, dermatitis, psoriasis and fungal disorders.

Vehicle

The vehicle of the presently disclosed sub-micron emulsion composition is preferably suitable for use in applications that require direct contact with human or animal skin.

The sub-micron emulsion composition is preferably in a form of a cream, foam, gel, lotion, pomade, mousse, balm, pump spray, aerosol spray, a wipe or any combinations thereof.

Preferred embodiments of the present disclosure include:

An oil in water sub-micron emulsion, said emulsion comprising an oil phase dispersed throughout a water phase, said emulsion comprising:
(a) at least one oil;
(b) at least one hydrophilic surfactant having an HLB greater than about 10;
(c) optionally at least one hydroxyl containing organic solvent; and
(d) water;

wherein the at least one surfactant is miscible both with water and the at least one hydroxyl containing organic solvent; and wherein the intensity weighted mean droplet diameter of the oil is less than 100 nm.

An oil in water sub-micron emulsion, said emulsion comprising an oil phase dispersed throughout a water phase, said emulsion comprising:
(a) at least one oil;
(b) at least one hydrophilic surfactant having an HLB greater than about 10;
(c) optionally at least one hydroxyl containing organic solvent comprising propylene glycol; and
(d) water;

wherein the at least one surfactant is miscible both with water and the hydroxyl containing organic solvent; and wherein the intensity weighted mean droplet diameter of the oil is less than 100 nm.

An oil in water sub-micron emulsion, said emulsion comprising an oil phase dispersed throughout a water phase, said emulsion comprising:
(a) at least one oil;
(b) at least one hydrophilic surfactant having an HLB greater than about 10;
(c) optionally at least one hydroxyl containing organic solvent comprising propylene glycol; and
(d) water;

wherein the at least one surfactant is miscible both with water and the hydroxyl containing organic solvent; and

wherein the intensity weighted mean droplet diameter of the oil remains below 100 nm for at least eight weeks when stored at 40° C.

An oil in water sub-micron emulsion, said emulsion comprising an oil phase dispersed throughout a water phase, said emulsion comprising:
(a) at least one oil;
(b) oleth-10;
(c) optionally propylene glycol; and
(d) water;

wherein the intensity weighted mean droplet diameter of the oil remains below 100 nm for at least eight weeks when stored at 40° C.

An oil in water sub-micron emulsion composition, comprising the sub-micron emulsion according to any one of the herein disclosed embodiments and at least one pharmaceutically active ingredient.

An oil in water sub-micron emulsion composition according to any one of the herein disclosed embodiments, wherein the at least one pharmaceutically active ingredient is hydrocortisone butyrate.

An oil in water sub-micron emulsion composition, said emulsion composition comprising an oil phase dispersed throughout a water phase, said emulsion composition comprising:
(a) at least one oil;
(b) oleth-10;
(c) propylene glycol;
(d) hydrocortisone butyrate; and
(e) water;

wherein the intensity weighted mean droplet diameter of the oil remains below 100 nm for at least eight weeks when stored at 40° C.

An oil in water sub-micron emulsion composition, said emulsion composition comprising an oil phase dispersed throughout a water phase, said emulsion composition comprising:
(a) between 1 and 10% by weight of at least one oil;
(b) between 3 and 12% by weight oleth-10;
(c) between 5 and 15% by weight propylene glycol;
(d) up to 0.5% by weight hydrocortisone butyrate; and
(e) between 60 and 90% by weight water.

An oil in water sub-micron emulsion composition, said emulsion composition comprising an oil phase dispersed throughout a water phase, said emulsion composition comprising:
(a) between 1 and 10% by weight of at least one oil;
(b) between 3 and 12% by weight oleth-10;
(c) between 5 and 15% by weight propylene glycol;
(d) up to 0.5% by weight hydrocortisone butyrate;
(e) between 60 and 90% by weight water;
(f) up to 1% by weight buffer;
(g) between 2 and 10% by weight humectant;

wherein the pH of the composition is less than or equal to 5.0.

An oil in water sub-micron emulsion composition, said emulsion composition comprising an oil phase dispersed throughout a water phase, said emulsion composition comprising:
(a) between 1 and 10% by weight of at least one oil;
(b) between 3 and 12% by weight oleth-10;
(c) between 5 and 15% by weight propylene glycol;
(d) up to 0.5% by weight hydrocortisone butyrate; and
(e) between 60 and 90% by weight water;

wherein the intensity weighted mean droplet diameter of the oil remains below 100 nm for at least eight weeks when stored at 40° C.

An oil in water sub-micron emulsion aerosol foam composition said composition comprising:
(a) at least one active pharmaceutical ingredient; and
(b) an oil in water sub-micron emulsion foam comprising an oil phase dispersed throughout a water phase, said oil in water sub-micron emulsion foam composition comprising:
(i) at least one oil;
(ii) at least one hydrophilic surfactant having an HLB greater than about 10;
(iii) at least one hydroxyl containing organic solvent; and
(iv) water;

wherein the at least one surfactant is miscible both with water and the at least one hydroxyl containing organic solvent; and

wherein the at least one active pharmaceutical ingredient comprises hydrocortisone butyrate.

A oil in water sub-micron emulsion aerosol foam composition said composition comprising:
(a) at least one active pharmaceutical ingredient; and
(b) an oil in water sub-micron emulsion foam comprising an oil phase dispersed throughout a water phase, said oil in water sub-micron emulsion foam composition comprising:
(i) at least one oil;
(ii) at least one hydrophilic surfactant having an HLB greater than about 10;
(iii) at least one hydroxyl containing organic solvent comprising propylene glycol; and
(iv) water;

wherein the at least one surfactant is miscible both with water and the at least one hydroxyl containing organic solvent; and

wherein the at least one active pharmaceutical ingredient comprises hydrocortisone butyrate.

In any one or more of the above disclosed embodiments the compositions or foams are substantially free of lipophilic surfactants.

In any one or more of the above disclosed embodiments the compositions or foams contain a single hydrophilic surfactant.

### EXAMPLES

The following Examples describe the emulsions and emulsion compositions according to the present disclosure and are intended to illustrate the disclosure. The Examples are not to be construed as limiting in any way the scope of the present disclosure.

Preparation of Sub-Micron Emulsion Composition

Hydrocortisone butyrate was dissolved in propylene glycol in a first vessel with stirring at ambient temperature. The remaining ingredients as listed under ‘oil phase’ in Table 1 were added and the resulting mixture heated to 70-75° C. In a second vessel the ingredients listed under ‘water phase’ were combined and heated to 70-75° C. The water phase was added to the oil phase. Stirring and heating was continued at 75-85° C. until the cloud point was reached. The mixture was then cooled to 40° C. and glycerine slowly added. The emulsion composition was then further cooled to 20-25° C. with stirring.

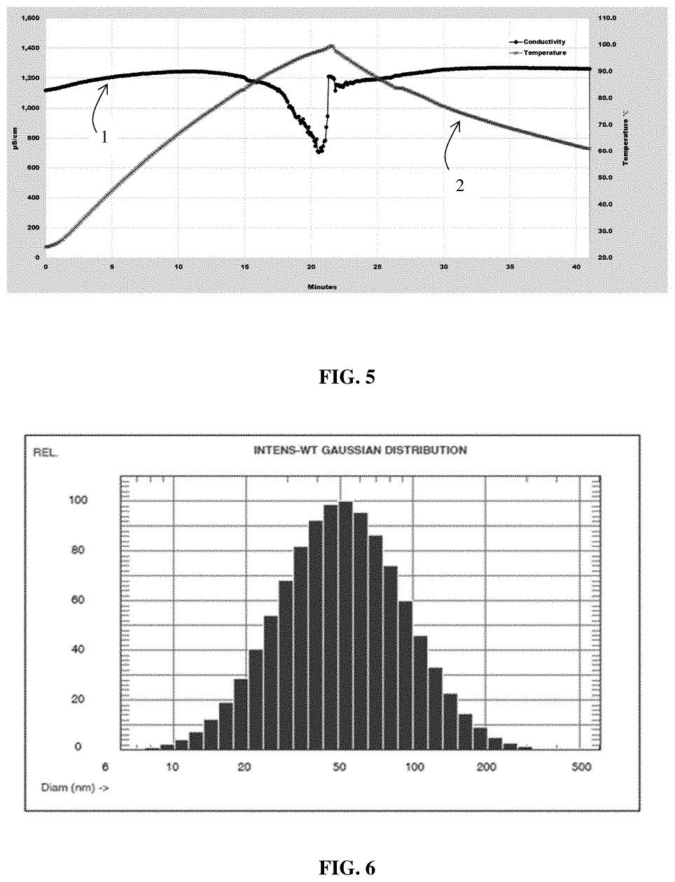

FIG. 5 illustrates conductivity in μS/cm (1) and temperature in C (2) vs time in minutes of the formulation during preparation. Conductivity measurements are ideally suited to determine the phase continuity of an emulsion. A water continuous emulsion will have conductivity typical of the water phase. An oil continuous emulsion will have negligible conductivity. A bicontinuous emulsion will have a conductivity intermediate between that of water and oil. See, for example, US 2010/0009063 (Beverungen et al) which teaches [0064] that in a phase inversion temperature (PIT) process utilizing both hydrophilic and lipophilic surfactants in which an oil in water emulsion inverts to a water in oil emulsion the conductivity drops to zero. FIG. 1 of Beverungen et al illustrates the relationship between conductivity and temperature and shows conductivity dropping to zero above the PIT temperature.

Referring to present FIG. 5, it can be seen that conductivity does not drop to close to zero as would be observed in a phase inversion temperature process that represents a water in oil emulsion and the observed conductivity is indicative of a bicontinuous emulsion.

Two sub-micron emulsion compositions were prepared using the above method and the relative amounts of components are shown in Table 1. Example 1 and Example 2 differ only in the amount of propylene glycol used. Sub-micron emulsion compositions were also prepared using 0.1% w/w hydrocortisone butyrate.

TABLE 1

| | | Example 1 | Example 2 |
|---|---|---|---|
| Ingredients | Grade | % w/w | % w/w |
| WATER PHASE | | | |
| Water (Deionised) | USP | 70.95 | 75.95 |
| Citric Acid Anhydrous | BP, USP | 0.10 | 0.10 |
| Sodium Citrate Dihydrate | BP | 0.10 | 0.10 |
| Disodium EDTA | BP | 0.20 | 0.20 |
| OIL PHASE | | | |
| Oleth-10 | USP | 7.50 | 7.50 |
| Light Mineral Oil | USP, BP | 5.00 | 5.00 |
| Butylated Hydroxytoluene | BP | 0.10 | 0.10 |
| Benzyl Alcohol | USP | 1.00 | 1.00 |
| Hydrocortisone Butyrate | PHA | 0.05 | 0.05 |
| Propylene Glycol | USP | 10.00 | 5.00 |
| FINAL PHASE | | | |
| Glycerine | USP, BP | 5.00 | 5.00 |
| TOTAL | | 100.00 | 100.00 |
| pH | | 4.32 | 4.20 |

Stability of Hydrocortisone Butyrate

The stability of hydrocortisone butyrate at ambient temperature (ca. 25° C.) and at 5° C. over a period of eight weeks was determined for the compositions of Examples 1 and 2 and the results are shown in Table 2.

TABLE 2

| | | Example 1 | | Example 2 | |
|---|---|---|---|---|---|
| | Storage | Hydrocortisone | Hydrocortisone | Hydrocortisone | Hydrocortisone |
| Time | Temp | Butyrate | Butyrate (%) | Butyrate | Butyrate (%) |
| Point | (° C.) | (% w/w) | relative to T = 0 | (% w/w) | relative to T = 0 |
| T = 0 | Ambient | 0.0648 | — | 0.0670 | — |
| T = 8 | 5 | 0.0659 | 101.7% | 0.0683 | 101.9% |
| weeks | Ambient | 0.0656 | 101.2% | 0.0682 | 101.8% |

The results indicate that there was no measurable change in the hydrocortisone butyrate concentration over an eight week period.

The sub-micron emulsion composition of Example 1 was also tested under accelerated stability conditions (40° C./75% RH) for up to eight weeks to determine the stability of hydrocortisone butyrate. Stability specification requirements for hydrocortisone butyrate are 90-110% Label Claim at two years shelf life. The results are shown in Table 3.

TABLE 3
Stability Data under accelerated conditions for Example 1 (pH 4.29)

| | Example 1 | | |
|---|---|---|---|
| | Storage | | Hydrocortisone |
| | Temp | Hydrocortisone | Butyrate (%) relative to |
| Time Point | (° C.) | Butyrate (% w/w) | T = 0 |
| T = 0 | Ambient | 0.0493 | — |
| T = 4 weeks | 5 | 0.0493 | 100.0% |
| | 25 | 0.0492 | 99.8% |
| | 40 | 0.0489 | 99.2% |
| T = 8 weeks | 5 | 0.0493 | 100.0% |
| | 25 | 0.0491 | 99.6% |
| | 40 | 0.0482 | 97.8% |

It is apparent that even at 40° C. the amount of hydrocortisone butyrate loss is extremely small.

Particle Size Distributions

The particle or droplet size of the sub-micron emulsion composition of Example 1 was determined at time zero and also after storage under varied conditions. The droplet size was measured on a particle sizer using dynamic light scattering.

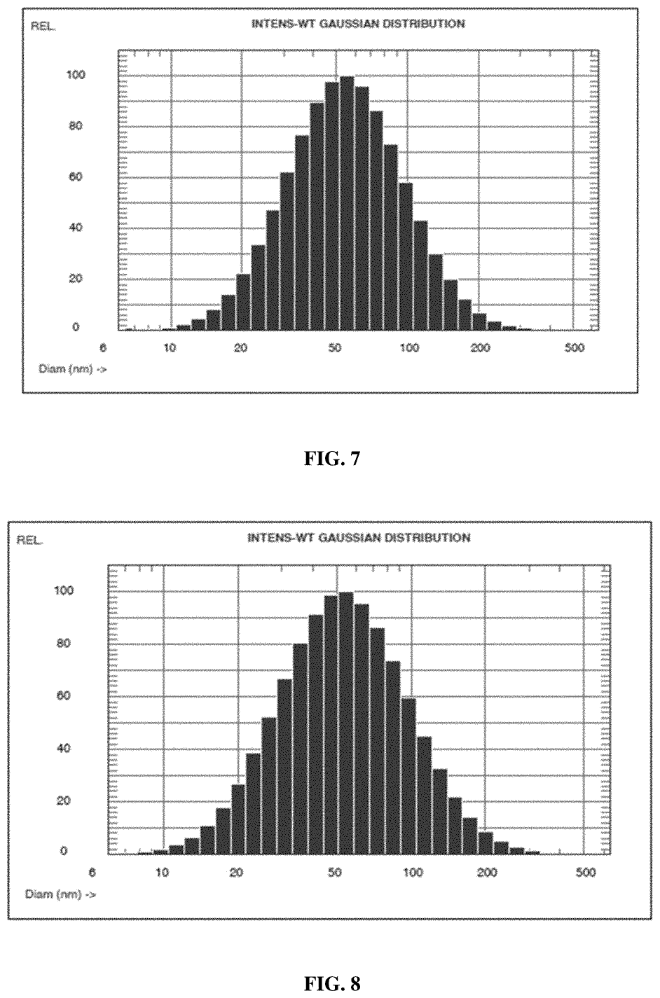

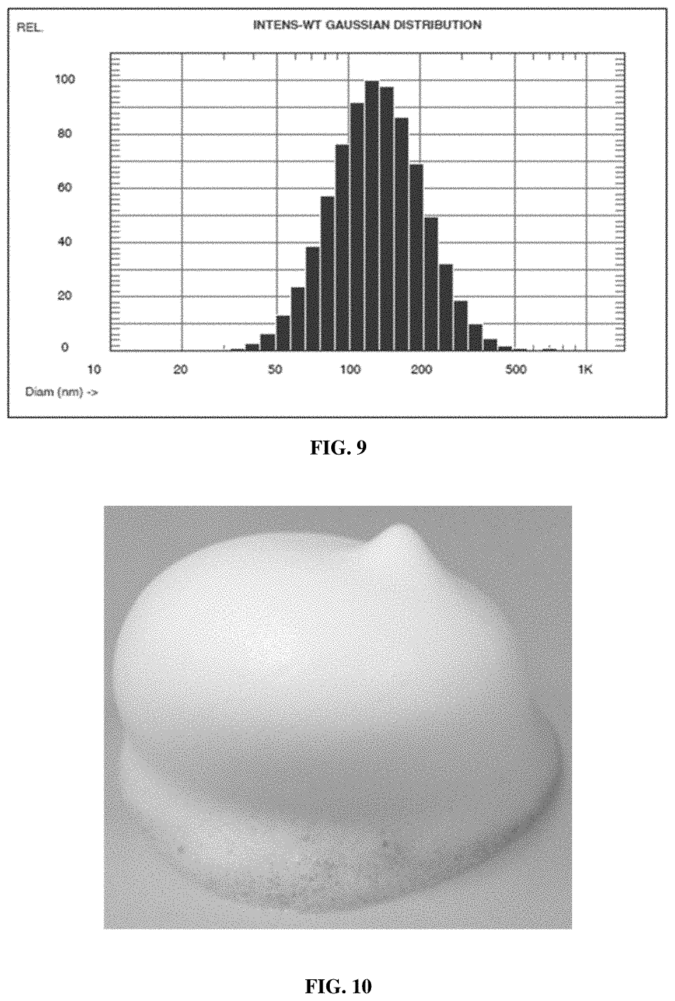

FIG. 6 shows the particle size distribution at time zero. The intensity weighted mean diameter of the droplets was 60.7 nm and 99% of the distribution was under 208 nm in diameter. FIG. 7 shows the particle size distribution after storing the emulsion at 5° C. for four weeks. There was very little change in the distribution with an intensity weighted mean diameter of 63.9 nm observed with 99% of the droplets under 204.7 nm in diameter. FIG. 8 shows the distribution after storing the emulsion at 25° C. for four weeks. There was very little change in the distribution with an intensity weighted mean diameter of 63.4 nm observed with 99% of the droplets under 213.8 nm in diameter. FIG. 9 shows the distribution after storing the emulsion at 40° C. for four weeks. There was very little change in the intensity weighted mean diameter of the droplets (65.1 nm). 99% of the droplets were under 372.5 nm in diameter.

Preparation of Foam

The sub-micron emulsion composition of Example 1 was packed in a can, fitted with a valve and pressurized with P75 propellant.

Foam assessment (see FIG. 10) indicated good foam peak upon expelling and easy to collapse upon manipulation on skin. The foams were non-greasy, non-sticky and fast absorbing. Furthermore, foam quality did not change after storage of the sub-micron emulsion composition for a period of one year in a PAM lined can.

Alternate Preparation of Sub-Micron Emulsion Compositions

The ingredients as listed under ‘oil phase’ in Table 1 with the exception of propylene glycol and hydrocortisone butyrate were combined and the resulting mixture heated to 70-75° C. In a separate vessel the ingredients listed under ‘water phase’ and including glycerine were combined and heated to 70-75° C. The water phase was added to the oil phase and stirring and heating was continued at 75-85° C. until the cloud point was reached. The mixture was then cooled to 40° C. and a solution of hydrocortisone butyrate in propylene glycol was added. The emulsion composition was then further cooled to 20-25° C. with stirring. The intensity weighted mean droplet size of the emulsion was 20 nm.

Alternate Preparation of Sub-Micron Emulsion Compositions

The ingredients as listed under ‘oil phase’ in Table 1 with the exception of propylene glycol and hydrocortisone butyrate were combined and the resulting mixture heated to 70-75° C. In a separate vessel the ingredients listed under ‘water phase’ and including glycerine were combined and heated to 70-75° C. In another vessel a solution of hydrocortisone butyrate in propylene glycol was prepared at ambient temperature. The heated water phase was added to the heated oil phase followed immediately by addition of the solution of hydrocortisone butyrate in propylene glycol. Stirring and heating was continued at 75-85° C. until the cloud point was reached. The mixture was then cooled to 20-25° C. with stirring.

The intensity weighted mean droplet size of the emulsion composition was 30 nm. After four weeks storage at 40° C. there were 1.55% by weight total degradants based on the staring amount of hydrocortisone butyrate.

Comparative Preparation of Sub-Micron Emulsion Compositions Using Lipophilic and Hydrophilic Surfactants

The ingredients as listed under ‘oil phase’ in Table 1, with the difference that the hydrophilic surfactant Oleth-10 was replaced with the hydrophilic surfactant Ceteareth-20 and lipophilic surfactant Oleth-5, were combined and the resulting mixture heated to 70-75° C. In a separate vessel the ingredients listed under ‘water phase’ and including glycerine were combined and heated to 70-75° C. The water phase was added to the oil phase and stirring and heating was continued at 75-85° C. until the cloud point was reached. The mixture was then cooled to 40° C. and a solution of hydrocortisone butyrate in propylene glycol was added. The emulsion composition was then further cooled to 20-25° C. with stirring.

The intensity weighted mean droplet size of the emulsion composition was 56 nm. After four weeks storage at 40° C. there were 0.89% by weight total degradants based on the starting amount of hydrocortisone butyrate.

Long Term Stability Results

The stability of formulations prepared as for Example 2 were determined over a period of one year at 25° C. Two formulations, Examples 3 and 4 containing respectively 0.05 and 0.1% w/w hydrocortisone butyrate, were studied.

The stability of two formulations having the same components as Example 2 but prepared using the method outlined in FIG. 4 were also determined over a period of one year at 25° C. Two formulations, Examples 5 and 6 containing respectively 0.05 and 0.1% w/w hydrocortisone butyrate, were studied. Table 4 collects the results.

TABLE 4

| | | | Hydrocortisone |
|---|---|---|---|
| | | Hydrocortisone | Butyrate |
| | | Butyrate | (% w/w) |
| | | (% w/w) | relative to T = 0 |
| Example 3 | 0 | 0.0526 | — |
| | 6 months | 0.0519 | 98.67% |
| | 12 months | 0.0514 | 97.72% |
| Example 4 | 0 | 0.1070 | — |
| | 6 months | 0.1052 | 98.32% |
| | 12 months | 0.1043 | 97.48% |
| Example 5 | 0 | 0.0524 | — |
| | 6 months | 0.0516 | 98.47% |
| | 12 months | 0.0510 | 97.33% |
| Example 6 | 0 | 0.1027 | — |
| | 6 months | 0.1009 | 98.25% |
| | 12 months | 0.0998 | 97.18% |

The stability of the same formulations (Examples 3 to 6) were determined over a period of six months at 40° C. The results are collected in Table 5.

TABLE 5

| | | | Hydrocortisone |
|---|---|---|---|
| | | Hydrocortisone | Butyrate |
| | | Butyrate | (% w/w) |
| | | (% w/w) | relative to T = 0 |
| Example 3 | 0 | 0.0526 | — |
| | 6 months | 0.0490 | 93.16% |
| Example 4 | 0 | 0.1070 | — |
| | 6 months | 0.0995 | 92.99% |
| Example 5 | 0 | 0.0524 | — |
| | 6 months | 0.0488 | 93.13% |
| Example 6 | 0 | 0.1027 | — |
| | 6 months | 0.0956 | 93.09% |

The sub-micron emulsion compositions of Examples 3 to 6 were packed in cans, fitted with valves and each was pressurized with P75 propellant. The pH of the foam was assessed over a period of 12 months. The results are summarized in Table 6.

TABLE 6

| Time | Temperature | pH | | | |
|---|---|---|---|---|---|
| Point | (° C.) | Example 3 | Example 4 | Example 5 | Example 6 |
| 0 | Ambient | 4.31 | 4.32 | 4.33 | 4.29 |
| 6 months | 25 | 4.25 | 4.21 | 4.52 | 4.48 |
| | 40 | 4.19 | 4.35 | 4.41 | 4.38 |
| 12 months | 25 | 4.11 | 4.11 | 4.25 | 4.18 |
| | 40 | 4.14 | 4.11 | 4.29 | 4.12 |

The results clearly indicate that stable emulsion compositions may be prepared using only hydrophilic surfactants. This is surprising based on prior art teachings. Further, long term stability of the active pharmaceutical ingredient is observed. Long term pH stability is also observed as is long term ability of stored emulsion compositions to provide stable foams.

All documents cited are herein fully incorporated by reference for all jurisdictions in which such incorporation is permitted and to the extent such disclosure is consistent with the description of the present disclosure.

Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1. An oil in water sub-micron emulsion, said emulsion comprising an oil phase dispersed throughout a water phase, said oil in water emulsion comprising:
(a) at least one oil;
(b) at least one hydrophilic surfactant having an HLB greater than about 10;
(c) optionally at least one hydroxyl containing organic solvent; and
(d) water;
wherein the at least one surfactant is miscible both with water and the at least one hydroxyl containing organic solvent.

2. An oil in water sub-micron emulsion composition, said composition comprising:
(a) at least one active pharmaceutical ingredient; and
(b) the oil in water sub-micron emulsion according to claim 1.

3. An oil in water sub-micron emulsion aerosol foam, said foam being actuated from the oil in water sub-micron emulsion according to claim 1 with a propellant.

4. An oil in water sub-micron emulsion aerosol foam composition, said composition comprising:
(a) at least one active pharmaceutical ingredient; and
(b) the oil in water sub-micron emulsion aerosol foam according to claim 3.

5. An oil in water sub-micron emulsion according to claim 1, wherein the emulsion is substantially free of a lipophilic surfactant.

6. A process for the preparation of an oil in water sub-micron emulsion according to claim 1, the process comprising at least the steps of heating a mixture of at least, water, at least one oil, and at least one hydrophilic surfactant, to a temperature above the cloud point of the hydrophilic surfactant and subsequently cooling the mixture to form an oil in water sub-micron emulsion.

7. A process according to claim 6, wherein the process does not comprise the step of phase inversion from an oil in water sub-micron emulsion to a water in oil sub-micron emulsion and does not comprise the step of phase inversion from a water in oil sub-micron emulsion to an oil in water sub-micron emulsion.

8. A process for the preparation of an oil in water sub-micron emulsion composition according to claim 2, the process comprising at least the steps of heating a mixture of at least, water, at least one oil, and optionally at least one hydrophilic surfactant, to a temperature above the cloud point of the hydrophilic surfactant and subsequently cooling the mixture to form an oil in water sub-micron emulsion and wherein the at least one pharmaceutically active agent is added before or after emulsion formation.

9. A process according to claim 8, wherein the process does not comprise the step of phase inversion from an oil in water sub-micron emulsion to a water in oil sub-micron emulsion and does not comprise the step of phase inversion from a water in oil sub-micron emulsion to an oil in water sub-micron emulsion.

10. A process according to claim 8, the process comprising:
(a) admixing at least one oil, at least one hydrophilic surfactant having an HLB greater than about 10 and water;
(b) heating the mixture of step a) to a temperature above the cloud point of the hydrophilic surfactant so as to form a bicontinuous emulsion;
(c) cooling the bicontinuous emulsion to form an oil in water sub-micron emulsion; and
(d) adding at least one active pharmaceutical ingredient optionally dissolved in at least one hydroxyl containing organic solvent to the sub-micron emulsion.

11. A process according to claim 8, the process comprising:
(a) heating a mixture of at least one oil, at least one hydrophilic surfactant having an HLB greater than about 10, optionally at least one hydroxyl containing organic solvent and at least one active pharmaceutical ingredient to a temperature above 25° C.;
(b) heating water to a temperature above 25° C.;
(c) combining the heated water with the heated oil mixture;
(d) heating the mixture of step c) to a temperature above the cloud point of the hydrophilic surfactant so as to form a bicontinuous emulsion; and
(e) cooling the bicontinuous emulsion to form an oil in water sub-micron emulsion.

12. A process according to claim 8, the process comprising:
(a) admixing at least one oil, at least one hydrophilic surfactant having an HLB greater than about 10 and water;
(b) heating the mixture formed in a) to a temperature above 25° C.;
(c) adding at least one active pharmaceutical ingredient optionally dissolved in at least one hydroxyl containing organic solvent to the heated mixture;
(d) heating the mixture formed in c) to a temperature above the cloud point of the hydrophilic surfactant so as to form a bicontinuous emulsion; and
(e) cooling the bicontinuous emulsion to form an oil in water sub-micron emulsion.

13. A process according to claim 4, the process comprising:
(a) heating a mixture of at least one oil, at least one hydrophilic surfactant having an HLB greater than about 10, optionally at least one hydroxyl containing organic solvent and at least one active pharmaceutical ingredient to a temperature above 25° C.;
(b) heating water to a temperature above 25° C.;
(c) combining the heated water with the heated oil mixture;
(d) heating the mixture of step c) to a temperature above the cloud point of the hydrophilic surfactant so as to form a bicontinuous emulsion;
(e) cooling the bicontinuous emulsion so as to form an oil in water sub-micron emulsion composition; and
(f) actuating a sample of the sub-micron emulsion composition with a propellant to form an oil in water sub-micron emulsion aerosol foam composition.

14. An oil in water sub-micron emulsion composition according to claim 2, wherein the at least one active pharmaceutical ingredient is soluble in the at least one hydroxyl containing organic solvent and/or is insoluble or sparingly soluble in water and wherein the at least one active pharmaceutical ingredient is selected from the group consisting of corticosteroids, particularly hydrocortisone butyrate, antihistamines, antibacterial agents, antifungal agents, antiviral agents, antiseptics, sunscreens, anaesthetics, analgesics, and skin conditioning agents, salts thereof, derivatives thereof and mixtures thereof.

15. An oil in water sub-micron emulsion according to claim 1, wherein 99% of oil droplets in the sub-micron emulsion have an intensity weighted mean diameter less than 500 nm or less than 400 nm, after storage of the emulsion at 40° C. for eight weeks.

16. An oil in water sub-micron emulsion composition according to claim 2, wherein the amount of at least one active pharmaceutical ingredient in the sub-micron emulsion composition decreases by less than 10 wt. %, when the composition is stored at 40° C. for eight weeks.

17. An oil in water sub-micron emulsion according to claim 1, wherein the emulsion comprises a single surfactant.

18. An oil in water sub-micron emulsion according to claim 1, wherein the emulsion, has a pH less than or equal to 7.0, or less than or equal to 6.5, or less than or equal to 6.0, or less than or equal to 5.5, or less than or equal to 5.0.

19. A method of treating a disease, disorder or condition, comprising administering to the skin of a subject requiring such treatment an effective amount of a sub-micron emulsion composition according to claim 2.

20. A method according to claim 19, wherein the skin disease, disorder or condition is selected from the group consisting of acne, rosacea, dermatitis, psoriasis and fungal disorders.
